(19) United States
(12) Patent Application Publication
(10) Pub. No.: US 2002/0058704 A1
(43) Pub. Date: May 16, 2002

(54) Multicomponent compositions containing chitosan and methods of preparing same

(75) Inventor: Sohail Malik, Roswell, GA (US)

Correspondence Address: KIMBERLY-CLARK WORLDWIDE, INC., 401 NORTH LAKE STREET, NEENAH, WI, 54956

(73) Assignee: Kimberly-Clark Worldwide, Inc.

(21) Appl. No.: 10/012,999

(22) Filed: Nov. 13, 2001

Related U.S. Application Data
(60) Provisional application No. 60/249,038, filed on Nov. 15, 2000.

(51) Int. Cl. C07C 61/08; C07C 61/00; A61K 31/19

(52) U.S. Cl. 514/557; 424/401; 562/400

(57) ABSTRACT

A method of preparing multicomponent salts of a compound in aqueous solution includes the steps of first selecting at least a weak acid, then selecting at least a first weak base, mixing an equivalent mole amount of the weak acid with the weak base in water to form an aqueous solution, adding at least a second weak base, to the solution and, finally, mixing the aqueous solution to yield a multicomponent salt composition.

### FIELD OF INVENTION

The present invention relates to salts of compounds, including chitosan, and methods of preparing the salts. In particular, the methods of preparing the salts and the resulting product salts can be used for various applications, including those in the pharmaceutical, cosmetic and nutritional areas.

### BACKGROUND OF INVENTION

Chitosan is a deacetylated product of chitin (C₈H₁₃NO₅)ₙ, an abundant natural glucosamine polysaccharide found in the ecosystem. In particular, chitin is found in the shells of crustaceans, such as crabs, lobsters and shrimp. The compound is also found in the exoskeletons of marine zooplankton, in the wings of certain insects, such as butterflies and ladybugs, and in the cell wall of yeasts, mushrooms and other fungi.

On the structural level, chitosan is predominantly polyglucosamine, and is generally prepared by the alkaline hydrolysis of chitin. The degree of deacetylation normally ranges from 70-98%. The deacetylated amino groups, at a pH below about 6 are protonated, and therefore are responsible for positive charges, which make the chitosan polymer soluble in water. This characteristic also leads to high positive charge density in the chitosan compound.

In addition to being non-toxic, biocompatible and biodegradable, chitosan is also reported in the scientific literature to possess hemostatic, antimicrobial properties and other biomedical attributes. See for instance, Rev Macromol. Chem Phys., C40, 69-83 (2000), Chitin and Chitosan, Editors, G. Skjak-Braek, T. Anthonsen and P. Sanford, Elsevier, (1988); Chitin in Nature and Technology, Editors, R. Muzzarelli, C. Jeuniaux and G. W. Gooday, Plenum Press, (1986).

The biocompatibility of chitosan administered orally and intravenously has been assessed in animals. Its LD₅₀ is over 16 g/Kg in mice, which is higher than for sucrose. LD₅₀ is traditionally defined as the median lethal dose of a substance, which will kill 50% of the animals receiving that dose, with the dose being calculated on amount of material given per gram or kilogram of body weight, or amount per unit of body surface area. See for instance, the 18^{th} Edition of Taber's Cyclopedic Medical Dictionary, p. 1085. The hemostatic properties of Chitosan have also been evaluated in the scientific literature in publications such as Ann. Thor. Surg., 35, 55-60, (1983); J Oral Maxillof Surg, 49, 858-63, (1991).

In recent years, however, attention has been directed in the research community towards biomedical applications of the chitosan compound. In this regard, the use of chitosan in the pharmaceutical and healthcare industry is currently being evaluated. For instance, use of chitosan has been reported in a pharmaceutical product in Pharm Res, 15, 1326-31, (1998). The use of chitosan in the pharmaceutical industry as an excipient has also been explored in Pharm Res, 15, 1326-31, (1998) and Drug Dev. Ind Pharm, 24, 979-93, (1998).

Antimicrobial properties of chitosan have been reported against Gram positive and Gram negative bacteria, including Streptococcus spp., Staphylococcus aureus, Staphylococcus epidermidis, Staphylococcus haemolyticus, Pseudomonas, Escherichia, Proteus, Klebsiella, Serratia, Acinobacter, Enterobacter and Citrobacter spp. See for instance, Muzzarelli et al., in Industrial Polysaccharides: Biomedical and Biotechnological Advances, Eds., V. Crescenzi and S. S. Stivala, Gordon and Breach, pp. 77-88, (1990) and Antimicr. Agents Chemoth., 34, 2019-24, (1990).

Chitosan has also been described in the literature to induce repair of tissue containing regularly arranged collagen bundles. See for instance Biomaterials, 9, 247-52, (1988). Additionally, non-woven fabrics made of chitosan fibers have been developed. See for instance, Eur. J. Plastic Surg., 10, 66-76, (1987).

Further, chitin and chitosan derivatives have been studied for their antitumor effects. See for instance, Carbohydr. Res, 151, 403-8, (1986); and Chem. Pharm, 36, 784-90, (1988). Chitosan has additionally been reported as an effective immunomodulator in Vaccine, 4, 151-6, (1986); and K. Nishimura in Chitin Derivatives in Life Sciences, Ed., S. Tokura, Japan Chitin Soc., (1992).

The use of chitosan in foods has also been reported in Proc Chim Aerosol Sel, 28, 5-8, (1987); Shipin Kexue, (Beijing), 87, 6-9, (1987); and An Asoc Quim Argent, 86, 1-4, (1998).

Finally, numerous chitosan salts have been reported in the literature to improve the properties of chitosan, i.e. Chitin and Chitosan, Editors, G. Skjak-Braek, T. Anthonsen and P. Sanford, Elsevier, (1988); Chitin in Nature and Technology, Editors, R. Muzzarelli, C. Jeuniaux and G. W. Gooday, Plenum Press, (1986); and U.S. Pat. No. 2,040,879 to Rigby for Substantially Undergraded Deacetylated Chitin and Process for Producing the Same; and U.S. Pat. No. 2,040,880 also to Rigby, for Process For the Preparation of Films and Filaments and Products Thereof.

Nevertheless, despite all of the research paths which have been pursued in the study of chitosan, as a result of the basic pH nature of chitosan, it has proven difficult to make salts of chitosan and other basic compounds without significantly changing the chemistry of the chitosan molecule. For instance, U.S. Pat. No. 4,971,956 to Suzuki et al. has described the difficulty in modifying chitosan to produce an appropriate water-soluble form, disclosing that water-insoluble forms are impractical for therapeutic application.

As has already been stated, chitosan is a natural polymer that is basic in nature. At a pH lower than about 6.3, the amines in the polymer become protonated and result in water-soluble chitosan. Once the chitosan polymer is protonated at a lower pH, it can be deprotonated by increasing the pH to above 6.5 by adding a basic compound. Therefore, it would not be practical to add or react basic compounds with chitosan without changing its solubility. This limits the ability to prepare any water-soluble chitosan salts with basic compounds. Further, there are several pharmaceutically active compounds which are basic in nature and therefore their delivery by chitosan would not be practical.

Therefore, there is a need for multicomponent water-soluble salts of chitosan, which can then be used for the previously described pharmaceutical, cosmetic and/or nutritional material applications. Further, there is a need for methods of synthesizing multicomponent water-soluble salts of chitosan. There is also a need for multicomponent water-soluble salts of other pharmaceutically, cosmetic and nutritional materials which are not easily made soluble without altering their respective chemical structure, or the use of covalent bonds. It is to the provision of such compositions and methods that the present invention is directed.

### SUMMARY OF THE INVENTION

A method of preparing multicomponent salts of a compound in aqueous solution includes the steps of first selecting at least a weak acid, then selecting at least a first weak base, mixing an equivalent mole amount of the weak acid with the weak base in water to form an aqueous solution, adding at least a second weak base to the solution and, finally, mixing the aqueous solution to yield a multicomponent salt composition.

### BRIEF DESCRIPTION OF THE DRAWINGS

FIG. 1 is a chart showing Rates of Re-epithelization in a rat model of wound healing, using a chitosan composition produced in accordance with the present inventive method.

### DETAILED DESCRIPTION OF THE INVENTION

As is known in the art, the union of an acid and base leads to the formation of a salt as part of a neutralization reaction. In the case of diacid and triacid bases, and of dibasic and tribasic acids, the mutual neutralization may vary in degree, producing respectively basic, neutral, or acid salts. A method for synthesizing water-soluble, multicomponent salts of compounds such as polyamines has now been discovered, which includes reacting an acid with at least two bases, in water, one of which base is desirably a polyamine compound, and with the number of bases depending upon the type of acid used (i.e. acidity or pH), to produce multicomponent salts. In an alternative embodiment, such bases could include a monoamine. Further, a method for synthesizing water-soluble, multicomponent chitosan salts includes reacting an acid with at least two bases, in water, one of which is chitosan, the number of bases depending upon the type of acid used (i.e. acidity or pH), to produce multicomponent chitosan salts. The method enhances the functionality of chitosan without creating any covalent bonding. The resulting compounds can then be used to treat skin injury and to treat/prevent skin conditions.

Specifically, the method includes mixing two or more bases in aqueous solution with a bridging acid (an acidic molecule between two basic molecules), without creating any covalent bonding, or without significantly changing the chemistry of any of the reactants (no bond changes). For the purposes of this application, the term “covalent bonding” shall mean that bonding which occurs when electrons are shared by two atomic nuclei.

Desirably, in accordance with the method, one mole of an acid (e.g. a dicarboxylic acid) is first mixed with one mole of a base (e.g. a monoamine) and then mixed with a second base (e.g. chitosan or a basic drug molecule), to form a multicomponent water soluble salt composition, and in the case of chitosan, a multicomponent water soluble chitosan salt.

In a similar fashion, one mole of a base may be first mixed with one mole of an acid in water, and then mixed with another acid to form a multicomponent water soluble salt composition, with a bridging base. Chitosan salts prepared by these methods remain water-soluble as long as the acidity or pH of the solution is maintained less than about 6, desirably less than about 5.0.

Multifunctional, multicomponent salts of compounds can be produced by this approach, and such compounds can be used in the applications previously described. Desirably, weak acids should be used in the inventive methods. It should be recognized that at least for the purposes of this application, strong acids are those which completely dissociate in water to give H+ and an anion. Weak acids, on the other hand, partially dissociate in water to give H+ and an anion. Weak acids, for the purpose of this application may be exemplified by acids other than HC1, H₂SO₄, HNO₃, HClO₄, HBr and HI. Such weak acids include for example organic acids, acidic compounds having more than one acidic protons, and pharmaceutically active compounds. Weak acids useful in the inventive method can be selected for example from polycarboxylic acids, such as di, tri, and tetra-carboxylic acid, aspartic acid, glutamic acid, ascorbic acid, succinic acid, glutaric acid and chlorogenic acid. This list is not meant to be limiting in scope. Desirably such acid is selected from compounds having more than one acidic proton.

Desirably, weak bases should be used in the inventive method. For the purposes of this application, strong bases completely dissociate into an OH— ion and a cation. Weak bases do not furnish OH— ions by complete dissociation. They do however react with water to furnish OH— ions. With the exception of the hydroxides of Groups I and II of the periodic table, all other bases are generally weak. Such bases include for example monoamines and polyamines. Bases useful in the inventive method can be selected for example from glucosamine, mannosamine, galactosamine, caffeine, niacinamide, and benzamide. This list is not meant to be limiting in scope. Such acids and bases can be monomers, polymers, cosmetic materials, nutritional materials and pharmaceutically active materials.

For the purposes of this application, the terms “pharmaceutically active materials”, “pharmaceuticals”, “pharmaceutical compounds”, and “pharmaceutical materials” shall each mean drugs, medicinal and curative products, as well as ancillary products such as tonics, dietary supplements, vitamins, deodorants and the like.

For the purposes of this application the term “nutritional materials” shall mean any element or compound that is essential to the life and growth of plants or animals, either as such or as transformed by chemical or enzymatic reactions. For example, such materials may include proteins, carbohydrates and fats as well as vitamins, minerals, oxygen and water.

For the purpose of this application the term “cosmetic materials” shall mean any preparation in the form of a liquid, semi-liquid, paste or powder applied to the skin to improve its appearance, and for cleaning, softening or protecting the skin or its adjuncts. Examples of cosmetic materials include without limitation animal fats (lanolin), vegetable oils, waxes, alcohols, surfactants, UV blocking agents, phenylene diamine, aluminum chlorohydrate, FDC organic dyes, talc, essential oils, inorganic pigments, chlorophyllins, nitrocellulose lacquers, and steroid hormones.

The present invention including some of the various embodiments is further described by the following examples. Such examples however, are not to be construed as limiting in any way either the spirit or the scope of the present invention. For each of the examples the pH was measured using a Beckman 295, available from Beckman Instruments, Inc., Fullerton, Calif.

### EXAMPLE 1

Succinic acid (0.344 g, 0.0029 moles) obtained from Sigma Chemical Company of St. Louis, Mo., was mixed with niacinamide (0.356 g, 0.0029 moles) also obtained from Sigma Chemical, in 60 ml H₂O (pH of 3.81 at 20.8° C.). The solution was stirred for 30 min. and chitosan (0.5 g, deg. of deacetylation 78.8%, 0.0029 moles) obtained from Vanson Inc. of Redmond, Wash. was added to the solution. The solution was stirred for 3 hrs to give a clear solution (pH of 4.30 at 21.4° C.) of chitosan niacinamide succinate salt.

### EXAMPLE 2

Succinic acid (0.344 g, 0.0029 moles) was mixed with benzamide (0.353 g, 0.0029 moles) obtained from Sigma Chemical, in 60 ml H₂O (pH of 3.02 at 20.3° C.). The solution was stirred for 30 min. at which point benzamide was completely dissolved in the solution. Chitosan (0.5 g, deg. of deacetylation 78.8%, 0.0029 moles) was added to this solution and it was stirred for 3 hrs to give a clear solution (pH of 4.20 at 21.3° C.) of chitosan benzamide succinate salt.

### EXAMPLE 3

Niacinamide ascorbate (0.87 g, 0.0029 moles) (prepared by mixing equimolar amounts of niacinamide (Sigma Chemical) and ascorbic acid (Sigma Chemical) as reported earlier by C. W. Bailey et al., J Amer. Chem. Soc., 67, 1184-5, (1945), was dissolved in 60 ml H₂O (pH of 3.85 at 20.9° C.). The solution was stirred for ten minutes and chitosan (0.5 g, deg. of deacetylation 78.8%, 0.0029 moles) was added to the solution. The solution was stirred for 3 hrs to give a clear solution (pH of 4.62 at 21.4° C.) of chitosan niacinamide ascorbate salt.

### EXAMPLE 4

### Pharmaceutical Application

### Accelerated Wound Healing by Chitosan Niacinamide Ascorbate

This example illustrates the ability of one of the compounds produced in accordance with the invention to accelerate wound healing in a rat model, as described in J. M. Davidson, Arch Dermatol Res., 290 (Suppl): S1-S11, 1998; J. P. Heggers et al., J Altern Compl Med., 2, 271-77, 1996; J. A. Hokanson et al., Wounds, 3, 213-220, 1991, which describe such testing protocols, and which are incorporated by reference herein in their entirety.

In particular, twelve albino rats (6M/6F), each weighing between 200-300 g, were anesthetized (90 mg/Kg Ketamine HCL and 10 mg/Kg Xylazine) and the entire dorsal region was shaved. Two wounds measuring 1 cm² were made on the dorsal skin, one on either side of the vertebral column, with a rotary dermabrasion device (Dermatome). One wound on each side was exposed to the test compound (Chitosan niacinamide ascorbate of example 3), where the pH was adjusted to 5.6 by adding chitosan (0.4 g) in 20 ml water. The material was filtered using Whatman qualitative filter paper and freeze-dried before application. The compound was applied topically by covering the entire wound. The other wound on each animal was covered with a sterile pad devoid of exogenous therapeutic material and served as an untreated control. The control and test materials were changed and applied once daily.

Four rats (2M/2F) were sacrificed at 48, 96 and 168 hours by carbon dioxide inhalation. Wound size was measured prior to the treatment (time 0), and at each time point as above. Cross sections were cut encompassing the entire wound and began at the margin of the initial wound and proceeded in 2.5 mm increments across the width of the wound. The epithelial thickness of the three sections of the wound (margin, center and midpoint between these two) was measured by morphometric analysis of the microscopic image using Image-Pro Plus software, Version 3.0 (Media Cybernetics). The average thickness of these sites within the wound was determined for wound healing of the entire site.

At the 48 hour data point, the control wound site had a mean epithelial thickness of 16.9±4.5 μm (mean±SEM) compared to the mean thickness of 40.9±5.8 μm at the test site (p<0.05). At the 96 hour data point, the epithelial thickness was 80.1±7.1 μm and 33.4±5.5 μm for test and control sites, respectively (p<0.05). The final data point at 168 hours revealed significantly greater epithelization of the test sites compared to control sites with epithelial thickness of 121.9±11.1 μm and 68.9±4.1 μm, respectively (p<0.05).

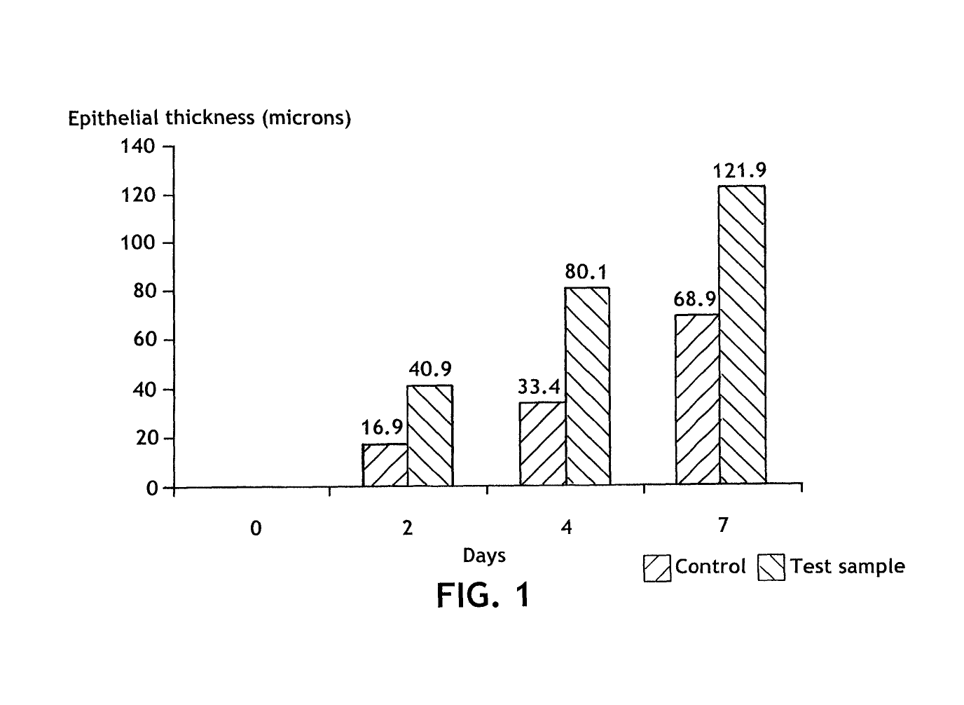

The rate of epithelization at the test and control sites was plotted vs time, and the results are shown in FIG. 1. The rate of epithelization at the test sites was greater than that of the control sites, demonstrating wound healing in the presence of the test compound compared to the control. Based on the slope of the lines between data points on the chart, there was a 93% increase in the rate of epithelization at the test sites compared to the control sites. In particular, the rate of reepithialization by test samples on day 2 and day 4 was more than double the rate of reepithialization of the control samples.

While the invention has been described in detail with particular reference to a preferred embodiment thereof, it should be understood that many modifications, additions, and deletions can be made thereto without departure from the spirit and the scope of the invention as set forth in the following claims.

I claim:

1. A method of preparing multicomponent salts of a compound in aqueous solution comprising the steps of:
a) selecting at least a weak acid;
b) selecting at least a first weak base;
c) mixing an equivalent mole amount of the weak acid with the weak base in water to form an aqueous solution;
d) adding at least one additional weak base to said solution; and
e) mixing the aqueous solution to yield a multicomponent salt composition.

2. The method of claim 1 wherein said weak acid is mixed with said first weak base to attain a solution pH lower than 5.

3. The method of claim 1 wherein said additional weak base is added to the solution, and the pH of the solution remains below 6.

4. The method of claim 1 wherein a base is a pharmaceutically active material.

5. The method of claim 1 wherein a base is a cosmetic material.

6. The method of claim 1 wherein a base is a nutritional material.

7. The method of claim 1 wherein said additional weak base is added to the solution in an equivalent mole amount to said first weak base.

8. A method of preparing multicomponent salts of a compound in aqueous solution comprising the steps of:
a) selecting at least a weak base;
b) selecting at least a first weak acid;
c) mixing an equivalent mole amount of the first weak acid with the weak base in water to form an aqueous solution;
d) adding at least one additional weak acid to the solution; and
e) mixing the aqueous solution to yield a multicomponent salt composition.

9. The method of claim 8 wherein said first weak acid is mixed with said weak base to attain a solution pH lower than 5.

10. The method of claim 8 wherein said additional weak acid is added to the solution and the pH of the solution remains below 6.

11. The method of claim 8 wherein said additional weak acid is added to the solution in an equivalent mole amount to said first weak acid.

12. A method of preparing multicomponent salts of polyamines in aqueous solution comprising the steps of:
a) selecting at least a weak acid;
b) selecting at least a first weak base;
c) mixing an equivalent mole amount of the acid with the base in water to form an aqueous solution;
d) adding a polyamine to the solution; and
e) stirring the solution until clear to yield a multicomponent polyamine salt composition.

13. The method of claim 12 wherein said polyamine is added to the solution in an equivalent mole amount to said first weak base.

13. Multicomponent salt compositions of polyamines produced by the method of claim 12.

14. The method of claim 12 wherein said polyamine is chitosan.

15. The method of claim 12 wherein said acid is selected from the group consisting of polycarboxylic acids including di, tri, and tetra-carboxylic acids, aspartic acid, glutamic acid, ascorbic acid, succinic acid, glutaric acid and chlorogenic acid.

16. The method of claim 12 wherein said first base is selected from the group consisting of from glucosamine, mannosamine, galactosamine, caffeine, niacinamide, and benzamide.

17. The method of claim 12 wherein said acid is selected from compounds having more than one acidic proton.

18. The method of claim 12 wherein said acid is a pharmaceutically active material.

19. The method of claim 18 wherein said acid is ascorbic acid.

20. The method of claim 17 wherein said acid is a cosmetic material.

21. The method of claim 17 wherein said acid is a nutritional material.

22. The method of claim 12 wherein said acid is mixed with said first base to attain a solution pH lower than 5.

23. The method of claim 12 wherein said polyamine is added to the solution and the solution pH remains below 6.

24. A method of preparing multicomponent salts of polyamines in aqueous solution comprising the steps of:
a) selecting at least a weak acid;
b) selecting at least a first weak base;
c) mixing an equivalent mole amount of the acid with the base in water to form an aqueous solution;
d) adding a polyamine to the solution; and
e) stirring the solution until clear to yield a multicomponent polyamine salt composition.
f) treating a wound with the multicomponent salt composition.
